(19) United States
(12) Patent Application Publication
(10) Pub. No.: US 2004/0138099 A1
(43) Pub. Date: Jul. 15, 2004

(54) Insulin administration regimens for the treatment of subjects with diabetes

(76) Inventor: Eberhard Kurt Draeger, Munich (DE)

Correspondence Address: NOVO NORDISK PHARMACEUTICALS, INC, 100 COLLEGE ROAD WEST, PRINCETON, NY, 08540, US

(21) Appl. No.: 10/721,710

(22) Filed: Nov. 25, 2003

Related U.S. Application Data
(60) Provisional application No. 60/431,852, filed on Dec. 9, 2002.

(30) Foreign Application Priority Data
Nov. 29, 2002 (DK) PA 2002 01845

(51) Int. Cl. A61K 38/28

(52) U.S. Cl. 514/3

(57) ABSTRACT

The present invention relates to the treatment of subjects with diabetes and in particular, to insulin administration regimens that improve the quality of metabolic control in subjects with diabetes.

### CROSS-REFERENCE TO RELATED APPLICATIONS

This application claims priority under 35 U.S.C. 119 of Danish application no. PA 2002 01845 filed Nov. 29, 2002 and U.S. provisional application No. 60/431,852 filed Dec. 9, 2002 the contents of which are fully incorporated herein by reference.

### FIELD OF THE INVENTION

The present invention relates to the treatment of subjects with diabetes and in particular, to insulin administration regimens that improve the quality of metabolic control in subjects with diabetes.

### BACKGROUND OF THE INVENTION

A common treatment regimen for diabetes is the so-called basal-bolus therapy regimen. In conventional basal-bolus therapy regimens, short-acting insulin is injected prior to meals (“bolus”) and intermediate or long-acting insulin is injected once or twice daily (or sometimes even more frequently) to cover the basal insulin requirements. However, while this treatment regimen has been proven successful in reducing the incidence of late diabetic complications as shown in the Diabetes Control and Complication Trial (DCCT) [Diabetes Control and Complication Trial Research Group. The Effect of Intensive Treatment of Diabetes on the Development and Progression of Long-term Complications in Insulin-Dependent Diabetes Mellitus. N Enql J Med (1993); 392(14): 977-986.], the regimen exhibits an inherent source of variation with respect to the effect of insulin on the subject.

This “regimen-induced” variation is due to the fact that the events to which insulin administrations are linked (meals, waking up in the morning and going to bed in the evening) in conventional basal-bolus therapy regimens can vary widely for a given subject thereby resulting in day-to-day variations in the time periods between bolus and basal injections for a subject.

For example, if one considers the evening administrations of bolus and basal insulin before dinner and bedtime respectively, it is difficult to establish a stable insulin profile even intraindividually since people do not tend to have dinner or go to bed at the same time. Thus, the administration of the pre-dinner bolus and of the bedtime basal insulin occurs over rather variable time windows. This evening/nighttime variability is confounded by the fact that breakfast is very often not taken at a fixed time, which results in a variable basal insulin supply during the day.

Indeed, it has been observed that the nighttime blood glucose profile of the “gold-standard” of basal insulins, Neutral Protamine Hagedorn (“NPH”) insulin, shows a rather steep decline, with a minimum level around 5:00 am followed by an increase of blood glucose thereafter, indicating that NPH has a peak effect during the night (risk of hypoglycemia) and a too short duration of effect not allowing to achieve appropriate blood glucose values early in the morning (compensating for the dawn phenomenon).

Thus, this “regimen-induced” variation in insulin effect may negatively affect the quality of a subject's metabolic control and may also increase the patient's risk of hypoglycemia. This “regimen-induced” variation is independent of the characteristics of the applied basal insulin and is exacerbated by the fact that within a given patient, the pharmacokinetic profile, i.e. the systemic insulin concentration, is highly variable.

Therefore, there exists a need in the art for new dosing concepts that would take the regimen-induced variation out of the presently utilized conventional basal-bolus therapy regimens.

### SUMMARY OF THE INVENTION

The present invention relates to insulin administration regimens that allow an individual to reduce or avoid the “regimen-induced” variability in insulin effect described above. It is believed that the insulin administration regimens of the invention will result in an improvement in metabolic control (as measured by, for example, glycosylated hemoglobin or blood glucose levels) and/or a reduced risk of hypoglycemia.

In one embodiment, the present invention relates to an insulin administration regimen in which the basal insulin is administered at fixed times of day. Thus, where the basal insulin requirements are met by an insulin that is administered twice a day, the first and second daily doses of basal insulin are administered at a fixed time interval. The present invention therefore relates to a method for administering a daily dosage of basal insulin to a subject in need of such treatment, the method comprising:

a) administering to the subject a first dose of basal insulin at a first time point; and

b) administering to the subject a second dose of basal insulin at a second time point, wherein the second time point is at a fixed time interval after the first time point.

Alternatively, where the basal insulin requirements are met by an insulin that is administered once a day, the once daily administrations of basal insulin are given at or near the same time daily such that the interval between daily administrations is fixed. In this alternative embodiment, the invention provides a method for administering a once daily dose of basal insulin to a subject in need of such treatment, the method comprising:

a) administering to the subject a single dose of basal insulin at a specified time on day one; and;

b) repeating step a) on each successive day, where the single dose of basal insulin administered on each successive day is administered at about the same time as the basal insulin was administered on day one.

In another embodiment, the invention relates to an insulin administration regimen in which the time interval between administration of a dose of basal insulin and a mealtime dose of bolus insulin is fixed. In such an embodiment, the invention provides a method for administering insulin to a subject in need of treatment, the method comprising;

a) administering a mealtime dose of bolus insulin to the subject at a first time point; and;

b) administering a dose of basal insulin to said subject at a second time point, wherein the second time point is at a fixed time interval from the first time point.

In yet another embodiment, where a subject eats a meal at a fixed time (for example, dinner at6PM) daily, the invention relates to an insulin administration regimen in which the time interval between administration of the first and second doses of basal insulin is fixed as is the time interval between the second (ie evening) dose of basal insulin and the dose of bolus insulin administered at mealtime. The invention therefore provides a method for administering insulin to a subject in need of such treatment, the method comprising administering to the subject within a single day:

a) a first dose of basal insulin at a first time point;

b) a mealtime dose of bolus insulin at a second time point; and

c) a second dose of basal insulin at a third time point, wherein the second time point is at about the same time every day and wherein the time intervals between the first and third time points and the second and third time points respectively are fixed.

### DEFINITIONS

“Insulin” means human insulin, human insulin analogs, human insulin derivatives or mixtures thereof.

“Human insulin” means insulin having the amino acid sequence shown in D S H W Nicol and L F Smith: Amino-acid sequence of human insulin, Nature, (1960) 4736:483-485, hereby incorporated by reference where disulfide bonds exist between cysteines at the 6^{th} and 11^{th} positions of the A chain, between cysteines at the 7^{th} position of the A chain and the 7^{th} position of the B chain and between cysteins at the 20^{th} position of the A chain and the 19^{th} position of the B chain.

“Human insulin analog” means human insulin in which one or more amino acid residues have been deleted and/or replaced by other amino acid residues, including amino acid residues that are not encoded by the genetic code, or human insulin comprising an additional amino acid residue or amino acid residues.

“Human insulin derivative” means human insulin or a human insulin analog in which at least one organic substituent is bound to one or more of the amino acid residues of the insulin molecule.

“Meal” as used in the present application means breakfast, lunch, dinner or midnight snack.

“Mealtime” as used in connection with the administration of a dose of bolus insulin means that the bolus insulin is preferably administered from about 30 minutes before the meal starts to about 30 minutes after the meal is finished, more preferably from about 15 minutes before the meal starts until the meal is finished, and most preferably shortly before or at the beginning of the meal.

“Bolus insulin” as used in connection with administration of a dose of insulin means mealtime administration of a short-acting insulin.

“Basal insulin” as used in connection with administration of a dose of insulin means administration of intermediate or long-acting insulins.

### DESCRIPTION OF THE INVENTION

The present invention relates to novel insulin administration regimens designed to reduce or avoid the “regimen-induced” variability in insulin effect inherent in conventional basal-bolus therapy regimens. These novel regimens, may be used by any subject in need of such treatment where by “a subject in need of such treatment” is meant an individual with diabetes where diabetes includes, but is not limited to, type 1 and type 2 diabetes, gestational diabetes, diabetes resulting from genetic defects of β cell function or insulin action, diseases of the exocrine pancreas, endocrinopathies and/or infections, drug or chemical-induced diabetes, uncommon forms of immune-mediated diabetes and other genetic syndromes associated with diabetes.

It is believed that the insulin administration regimens of the present invention will result in an improvement in metabolic control and/or a reduced risk of hypoglycemia compared to the conventional bolus-basal therapy regimens.

Such improvement in metabolic control may be assessed through measurement of efficacy variables known to those of skill in the art including, but not limited, to glycosylated hemoglobin (HbA_{1c}), fasting plasma glucose (FPG), fasting blood glucose (FBG), ten point blood glucose profiles and 24 hour glucose profiles.

A reduced risk of hypoglycemia may be measured by the recording of a subject's hypoglycemic episodes over a given time period where a hypoglycemic episode may be classified as a blood glucose <2.8 mmol/l (50 mg/dl) and/or symptoms well known to be related to hypoglycemia. Such symptoms include, but are not limited to, cognitive dysfunction, irrational or aggressive behavior, amnesia and progressive confusion, convulsions and loss of consciousness, sweating, tremors, palpitations, blurring of vision, hunger and altered salivation, headache, dizziness, generalized weakness and parathesia.

Pharmaceutical compositions containing human insulin, a human insulin analog, a human insulin derivative or a mixture thereof for use in the methods of the present invention may be administered parenterally to subjects in need of such a treatment. Parenteral administration may be performed by subcutaneous, intramuscular or intravenous injection by means of a syringe, optionally a pen-like syringe. A further option is a composition which may be a powder or a liquid for the administration of human insulin, a human insulin analog, a human insulin derivative or mixtures thereof in the form of a nasal spray or by inhalation.

In a preferred embodiment, the human insulin, human insulin analogs or human insulin derivatives used in the present invention are administered subcutaneously, with preferred sites of administration including the abdomen, the thighs and the upper arms.

The insulin compositions for use in the invention can be prepared using the conventional techniques of the pharmaceutical industry which involves dissolving and mixing the ingredients as appropriate to give the desired end product.

Thus, according to one procedure, the human insulin or analog or derivative or mixture thereof is dissolved in an amount of water which is somewhat less than the final volume of the composition to be prepared. An isotonic agent, a preservative and a buffer is added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

Examples of isotonic agents are sodium chloride, mannitol and glycerol.

Examples of preservatives are phenol, m-cresol, methyl p-hydroxybenzoate and benzyl alcohol.

Examples of suitable buffers are sodium acetate and sodium phosphate.

A composition for nasal administration of an insulin analog according to the present invention may, for example, be prepared as described in European Patent No. 272097 (to Novo Nordisk A/S).

As the treatment regimens of the invention are intended for subjects having diabetes, the optimal dose level of the insulin compositions for any subject will depend on a variety of factors including the efficacy of the specific insulin composition employed, the age, body weight, physical activity, and diet of the subject, on possible combination with other drugs, and on the severity of the case of diabetes. It is recommended that the daily dosage of the composition be determined for each individual subject by those skilled in the art in a similar way as for known insulin compositions.

Examples of twice-a-day basal insulins for use in the methods of the inventor include any so-called intermediate insulins such as those described, for example, in U.S. Pat. Nos. 5,750,497 and 6,011,007. Such insulins may include, but are not limited to, NPH insulin, insulin derivatives such as Lys B29 (N∈-tetradecanoyl) des(B30) human insulin, and 30/70 mixtures of prompt insulin zinc (SemiLente®) with extended insulin zinc (Ultralente®), sold commercially as Lente®.

Examples of once-a-day basal insulins for use in the methods of the present invention include long-acting insulins such as those described, for example, in U.S. Pat. No. 5,656,722, where such insulins include insulin glargine (Lantus®) or extended insulin zinc (Ultralente®).

Examples of bolus insulin for use in the present invention include any insulin with a fast onset of effect and a short duration of effect. Examples of such rapid-acting insulins are described, for example, in the European patent applications having the publication numbers EP 214826, EP 375437 and EP 383472. Bolus insulins can include regular human insulin, human insulin analogs or human insulin derivatives or mixtures of any of the foregoing, such as a mixture of a human insulin and a human insulin analog. Examples of such insulins include, but are not limited to, human insulin, insulin lispro (Humalog®), insulin aspart (Novolog®), or a 30/70 mixture of insulin aspart and insulin aspart protamine (NovoMix®)

As now described in greater detail below, in the insulin administration regimens of the present invention, the subject is administered basal insulin once or twice daily supplemented by mealtime administrations of bolus insulin. However, unlike in the conventional basal-bolus therapy regimens where there is a “regimen-induced” variation in the time intervals between insulin administrations, the time intervals between the different insulin administrations in the regimens of the present invention are fixed.

In one embodiment, the present invention relates to an insulin administration regimen in which the doses of basal insulin are administered at fixed time intervals. Thus, for a basal insulin that requires twice-a-day administration, the present invention provides a method for administering a daily dosage of basal insulin to a subject in need of such treatment, the method comprising:

a) administering to the subject a first dose of basal insulin at a first time point; and

b) administering to the subject a second dose of the basal insulin at a second time point, wherein the second time point is at a fixed time interval after the first time point.

It is envisioned that the above method will be a permanent regimen in that it will be followed day to day by the subject. In the steady state situation, it is believed that the method will provide stable daily basal insulins profiles, which could then be supplemented by bolus insulin administration as needed. In addition, in the above method, as in the other methods of administration where the interval(s) between administrations are fixed, the time points of administration may be individualized so long as the fixed time intervals are kept constant. Thus in the methods of the invention, the patient would be free to move the insulin administration time points to another time of day provided the time interval between insulin administrations is kept constant.

For example in the above method for administering a daily dosage of twice-a-day basal insulin, the subject may change the administration of the first and second doses to accommodate his/her schedule, for example, to accommodate the timing of weekend activities. In such a situation, the administration of both doses is preferably shifted by less than one hour, more preferably by less than half an hour. However, if a shift of more than one hour is desired, a shift by one hour every second day is recommended in order to achieve a smooth transition between one time schedule to the next.

In the above method for administering the daily dosage of twice-a-day basal insulin, the time interval maybe fixed at 12 hours or at any time from between about 10 to about 14 hours, more preferably at any time from between about 11 to about 13 hours.

Of course, it is to be understood that the time interval chosen between the first and second daily doses of twice-a-day basal insulin would depend on the type of insulin blood concentration over time profile one desired to achieve. Thus, if one desired to achieve a flat insulin profile, one would fix the time interval at 12 hours; if one wanted to obtain higher levels of insulin while the subject was awake, the time interval would be set at less than 12 hours as described above; and if one wanted to obtain higher levels of insulin while the subject was asleep, one would fix the time interval at greater than 12 hours. Alternatively, one skilled in the art would recognize that variations from a flat insulin profile could also be achieved, as discussed below, by administering different doses of the basal insulin at the daily administration times.

It is to be further understood that in the above method and in the other methods of the invention involving administration of basal insulin, the individual administration of each dose of basal insulin should not vary more than about ±30 minutes from the scheduled time of administration, more preferably not more than about ±15 minutes from the scheduled time. Thus, if for example in the above method, the first dose of basal insulin was to be given at 8 AM and the second dose of basal insulin at 8 PM, the first dose could be administered from 7:30-8:30 AM and the second dose from 7:30-8:30 PM. It would therefore be understood by one skilled in the art that for the above example, if the time interval between the first and second doses is set at “about 12 hours”, the time interval may be 12 hours ±60 minutes, preferably 12 hours ±30 minutes.

In a preferred embodiment, the subject will select a convenient time in the morning for administration of the first dose of basal insulin, thereby allowing the second dose to be given in the evening.

It is further contemplated that the second dose of basal insulin may be taken before or after dinner but if taken before dinner it is preferred that there be no more than one hour between the basal dose and the dinnertime bolus dose.

In addition to providing constant daily insulin profiles, the above method also provides the flexibility to fix two different levels of basal insulin supply during the day by administering different doses at the two daily administration times. Thus, in the above method, the first and second doses of basal insulin may be the same or different depending on the subject's needs. Thus, where different levels of basal insulin may be desirable, the first and second doses of basal insulin may be different. In this embodiment, it is envisioned that because of meals during the day and the risk of hypoglycemia during the night, the first daily dose would typically be higher than the second daily dose.

In an alternative embodiment, where the basal insulin to be administered to the subject is intended for once-a-day administration, the invention provides a method for the administration of the single daily dose of basal insulin to a subject in need of such treatment, the method comprising:

a) administering to the subject a single dose of basal insulin at a given time on day one and;

b) repeating step a) on successive days where the single dose of basal insulin administered on day one is administered at about the same time on successive days.

In this embodiment, the daily doses of once a day insulin are administered at the same time ±30 minutes, preferably ±15 minutes every day. Thus, contrary to the teaching in the prior art that once-a-day insulin be administered before bedtime, a variable event, the present method contemplates administering the once-a-day insulin at a fixed time every day.

Preferably, the time of administration is after dinner but before bedtime.

In a preferred embodiment, the dose of once-a-day basal insulin to be administered is kept the same from day to day.

In another embodiment, the present invention provides an insulin administration regimen which is a method for administering insulin to a subject in need of such treatment, the method comprising:

a) administering a mealtime dose of bolus insulin to the subject at a first time point; and

b) administering a dose of basal insulin to the subject at a second time point,

wherein the second time point is at a fixed time interval from the first time point.

In this method, as in the previous method, the individual dose of the basal insulin should not vary more than ±30 minutes from the scheduled time of administration, more preferably not more than 35 15 minutes from the scheduled time.

In one embodiment, the mealtime for administration of the dose of bolus insulin is dinnertime.

It is to be understood that the basal dose of insulin administered in step (b) of the above method may be the second daily dose of basal insulin when the basal insulin is a twice-a-day basal insulin, or the single daily dose of basal insulin where the basal insulin is for once-a-day administration.

In the above method, the basal dose in step b) may be administered before or after the bolus dose to be administered in step a) where the time interval between the basal dose in step b) and the bolus dose in step a) would depend on the action profile (ie the duration of action and the shape of the insulin blood concentration versus time profile of the particular insulin to be administered) of the insulins to be administered.

In one embodiment, since the duration of action of basal insulins does not generally cover 24 hours, the basal insulin would be administered after the bolus insulin. When the dose of basal insulin in step b) is administered after the dose of bolus insulin to be administered in step (a), the time interval between administration of the doses of basal and bolus insulins is preferably a time selected from between about 3 to about 4 hours, more preferably at a time from between about 2 to about 3 hours where the bolus insulin is an insulin analog having a short duration of action.

Of course, one skilled in the art would understand that the optimal time interval between the dose of bolus insulin in step a) and the I dose of basal insulin in step b) would be based on the known pharmaceutical characteristics of the particular bolus insulin to be administered in step a) as well as its dose, where it is believed that the time interval will differ for human insulin and for insulin analogs with a fast onset of effect and a short duration of effect. In yet another embodiment, the invention relates to a method for administering insulin to a subject in need of much treatment, the method comprising administering to the subject within a single day:

a) a first dose of basal insulin at a first time point;

b) a mealtime dose of bolus insulin at a second time point; and

c) a second dose of basal insulin at a third time point,

wherein the second time point is at about the same time every day and wherein the time intervals between the first and third time points and the second and third time points respectively are fixed.

In this method, the time interval between the first and third time points and the second and third time points may be the same or different. In a preferred embodiment, the time intervals between the first and third time points and the second and third time points are different. In yet another preferred embodiment, the dose of bolus insulin in step b) is administered at dinnertime, preferably before dinner.

In one embodiment of the above method, the second dose of basal insulin is step c) is administered after the dose of bolus insulin in step b).

In this embodiment, the time interval between the first interval between the first and second doses of basal insulin (ie between the first and third time points) is fixed at about 12 hours or at a time between about 10 to 14 hours; preferably at a time from between about 11 to about 13 hours; and the time interval between the dose of bolus insulin (second time point) and the second dose of basal insulin (third time point) is selected at a time that is dependent on the action profile of the bolus insulin. In one embodiment, the time interval between the dose of bolus insulin (second time point) and the second dose of basal insulin (third time point) is between about 3 to about 4 hours.

In an alternative embodiment, the second dose of basal insulin in step c) may be administered before the dose of bolus insulin in step b). In this embodiment, the time intervals between the first and second doses of basal insulin and between administration of the dose of bolus insulin and the second dose of bolus insulin are again selected based on the actual profiles of the basal and bolus insulins to be administered.

Of course, it is to be understood that whether the second dose of basal insulin is administered before or after administration of the dose of bolus insulin in step (b), the first and second doses of basal insulin may be the same or different. Where the first and second doses of basal insulin are different, the first dose will preferably be larger than the second dose.

All scientific publications and patents cited herein are specifically incorporated by reference. The following example illustrates various aspects of the invention but is in no way intended to limit the scope thereof.

### EXAMPLES

### EXAMPLE 1

A three arm parallel group clinical trial of 16 weeks treatment comparing LysB29(N∈-tetradecanoyl) des(B30) human insulin administered at 12 hour intervals, LysB29(N∈-tetradecanoyl) des(B30) human insulin administered morning and bedtime and NPH insulin administered morning and bedtime in subjects with Type 1 diabetes. All subjects will receive insulin aspart (NovoRapid®) at meals. NPH insulin isn chosen as the comparator to LysB29(N∈-tetradecanoyl) des(B30) human insulin as it is the most frequently used basal insulin worldwide.

The insulins utilized in the trial are as follows:

Basal insulins

Human NPH Insulin (generic name: Human isophane insulin) Penfill® cartridges 3.0 ml 100 lU/ml; or

[LysB29(N∈-tetradecanoyl) des(B30) human insulin]Penfill® cartridges 3.0 ml 100 U/mI (100 U=2400 nmol).

bolus insulin

NovoRapid® Penfill® cartridges 3.0 ml 100 U/ml. (NovoRapid® is commercially available in the United States as NovoLog®).

Subjects will be dosed according to individual requirements for 16 weeks including an initial titration period. They will receive their basal dose of LysB29(N∈-tetradecanoyl) des(B30) human insulin or NPH insulin twice daily and NovoRapid® at meals. The injections will be given subcutaneously and the thigh or abdomen can be used as injection areas. The area chosen for each type of insulin should remain the same throughout the trial, and injection site should be rotated within the area chosen to prevent lipohypertrophy.

The objectives of the trial will be examined by the following efficacy and safety parameters:

Efficacy:

HbA_{1c} after 16 weeks of treatment (primary endpoint)

10-point blood glucose profile after 16 weeks of treatment

fasting plasma glucose (FPG lab) after 16 weeks of treatment

fasting blood glucose (FBG home) in the last 7 days of the treatment period

24-hours blood glucose profile within the last 4 weeks of the treatment period.

Safety:

Hypoglycaemic episodes during the last 12 weeks of treatment (nocturnal

23:00-06:00 and diurnal 06:00-23:00)

Adverse events, laboratory assessments (haematology, biochemistry, lipids), physical examination, ECG, fundoscopy/fundosphotography, weight and vital signs after 16 weeks of treatment

1. A method for administering a daily dosage of basal insulin to a subject in need of such treatment, said method comprising:
a) administering to said subject a first dose of basal insulin at a first time point; and
b) administering to said subject a second dose of said basal insulin at a second time point,
wherein said second time point is at a fixed time interval after said first time point.

2. The method of claim 1, wherein the time interval between the first and second doses of basal insulin is about 10 hours.

3. The method of claim 1, wherein the time interval between the first and second doses of basal insulin is about 12 hours.

4. The method of claim 1, wherein the time interval between the first and second doses of basal insulin is about 14 hours.

5. The method of claim 1, wherein the first and second doses of basal insulin are different.

6. The method of claim 1, wherein the first and second doses of basal insulin are the same.

7. The method of claim 1, wherein said basal insulin is a human insulin derivative.

8. The method of claim 7, wherein said human insulin derivative is LysB29 (N∈-tetradecanoyl) des(B30) human insulin.

9. The method of claim 1, wherein the basal insulin is administered subcutaneously.

10. The method of claim 1, wherein said subject has type 1 or type 2 diabetes.

11. A method for administering a once daily dose of basal insulin to a subject in need of such treatment, said method comprising:
a) administering to said subject a single dose of basal insulin at a specified time on day one; and
b) repeating step a) on each successive day, where the single dose of basal insulin ministered on each successive day is administered at the about the same time the basal insulin was administered on day one.

12. The method of claim 11, wherein the specified time at which the dose of basal insulin is administered is after noon.

13. The method of claim 12, wherein the specified time at which the basal insulin is administered is after dinner.

14. The method of claim 11, wherein the basal insulin is insulin glargine.

15. The method of claim 11, wherein the basal insulin is administered subcutaneously.

16. The method of claim 11, wherein said subject has type 1 or type 2 diabetes.

17. A method for administering insulin to a subject in need of treatment, said method comprising;
a) administering a mealtime bolus dose of insulin to said subject at a first time point; and;
b) administering a basal dose of insulin to said subject at a second time point,
wherein said second time point is at a fixed time interval from said first time point.

18. The method of claim 17, wherein said mealtime in step a) is dinnertime.

19. The method of claim 18, wherein said dose of bolus insulin is administered before dinnertime.

20. The method of claim 18, wherein the second time point is at a fixed time interval after said first time point.

21. The method of claim 20, wherein the time interval between the first and second time points is a time selected from between about 2 hours to about 4 hours.

22. The method of claim 18, wherein the second time point is at a fixed time interval before said first time point.

23. The method of claim 22, wherein the time interval between the first and second time point is a time selected from between about 2 hours to about 4 hours.

24. The method of claim 17, wherein the bolus insulin is human insulin.

25. The method of claim 17, wherein the bolus insulin is a human insulin analog.

26. The method of claim 25, wherein the bolus insulin is insulin lispro or insulin aspart.

27. The method of claim 17, wherein the basal insulin administered in step b) is a twice-a-day basal insulin.

28. The method of claim 27, wherein the dose of basal insulin administered at second time point in step b) is the second daily dose of basal insulin administered to said subject.

29. The method of claim 28, wherein the basal insulin is a human insulin derivative.

30. The method of claim 29, wherein the human insulin derivative is LysB29 (N∈-tetradecanoyl) des(B30) human insulin.

31. The method of claim 27, wherein the basal insulin administered to said subject is a first daily dose of basal insulin.

32. The method of claim 17, wherein the dose of basal insulin administered at the second time point in step b) is the single daily dose of basal insulin administered to said subject.

33. The method of claim 17, wherein the subject has type 1 or type 2 diabetes.

34. The method of claim 17, wherein the basal and bolus insulins are administered subcutaneously.

35. A method for administering insulin to a subject in need of such treatment; said method comprising administering to said subject within a single day:
a) a first dose of basal insulin at a first time point;
b) a mealtime dose of bolus insulin at a second time point; and
c) a second dose of basal insulin at a third time point,
wherein said second time point is at about the same time every day and wherein the time intervals between the first and third time points and the second and third time points respectively are fixed.

36. The method of claim 35, wherein the second dose of basal insulin in step c) is administered after the dose of bolus insulin in step b).

37. The method of claim 36, wherein the time interval between the second and third time points is a time selected from between about 2 hours to about 4 hours.

38. The method of claim 36, wherein the time interval between the first and third time points is about 12 hours.

39. The method of claim 36, wherein the mealtime dose of bolus insulin is administered at dinnertime.

40. The method of claim 35, wherein the first and second doses of basal insulin are the same.

41. The method of claim 35, wherein the first and second doses of basal insulin are different.

42. The method of claim 35, wherein the basal insulin is NPH human insulin.

43. The method of claim 35, wherein the basal insulin is a human insulin derivative.

44. The method of claim 43, wherein the human insulin derivative is LysB29 (N∈-tetradecanoyl) des(B30) human insulin.

45. The method of claim 35, wherein the bolus insulin is human insulin.

46. The method of claim 35, wherein the bolus insulin is a human insulin analog.

47. The method of claim 35, wherein the bolus and basal insulins are administered subcutaneously.

48. The method of claim 35, wherein the second dose of basal insulin in step (c) is administered before the bolus dose of insulin in step (b).

49. The method of claim 35, wherein the subject has type 1 or type 2 diabetes.

50. The method of claim 7, wherein said human insulin derivative is Lys^{B29}-(N^{∈}-(y-glutamyl-N^{α}-lithocholyl) des(B30) human insulin.

51. The method of claim 29, wherein said human insulin derivative is Lys^{B29}-(N^{∈}-(y-glutamyl-N^{α}-lithocholyl) des(B30) human insulin.

52. The method of claim 43, wherein said human insulin derivative is Lys^{B29}-(N^{∈}-(y-glutamyl-N^{α}-lithocholyl) des(B30) human insulin.
